(19) United States
(12) Patent Application Publication
(10) Pub. No.: US 2024/0376417 A1
(43) Pub. Date: Nov. 14, 2024

(54) NEW COMPOSITION BASED ON A SUPERABSORBENT POLYMER AND IRON FOR ACCELERATING THE DEGRADATION OF ORGANIC WASTE

(71) Applicant: APROTEK, SAINT-ROMAIN-LE-PUY (FR)

(72) Inventors: Stéphane DELHEUR, Saint Marcellin en Forez (FR); Christian CHAPELLE, Andrezieux Boutheon (FR)

(21) Appl. No.: 18/691,142

(22) PCT Filed: Sep. 9, 2022
(86) PCT No.: PCT/EP2022/075081

§ 371 Date: Mar. 12, 2024

(30) Foreign Application Priority Data
Sep. 17, 2021 (FR) FR2109799

(51) Int. Cl. C12M 1/12; C12M 1/00; C12M 1/107

(52) U.S. Cl. CPC C12M 25/16; C12M 21/04; C12M 23/20

(57) ABSTRACT

The present invention is intended for the technical field of organic waste fermentation and makes it possible to increase methane production yields by anaerobic fermentation from lignocellulose materials of plant origin. The invention concerns a new composition based on a superabsorbent polymer and iron for accelerating the degradation of this type of organic waste, as well as a process for using it.

The present invention is intended for the technical field of fermentation of waste of organic origin and allows for increasing the yields of methane production by anaerobic fermentation from lignocellulose materials of plant origin. The invention relates to a new composition based on a superabsorbent polymer and iron making it possible to accelerate the degradation of this type of organic waste as well as a process implementing it.

### TECHNICAL FIELD OF THE INVENTION

Lignocellulosic agricultural plant residues are among the slowest to be degraded during anaerobic digestion due to the presence of lignin. This is why these residues are very little valorized by fermentation even though they constitute an abundant and available biomass because they do not compete with food. There is therefore a real need to improve their biodegradation process in anaerobic digesters, particularly with a view to optimizing their degradation in methane production processes.

Methane is a gas that can be produced by the fermentation of biodegradable material, such as waste of organic origin, particularly agricultural, urban and agro-industrial waste. This biological process is called methanization. It consists of transforming, in the absence of oxygen, organic matter into:
a renewable energy, called biogas, which includes, among other things, methane (CH4), generally 50% to 70%, and carbon dioxide (CO2),
as well as a digestate that can be used as fertilizer.

The biogas thus produced can be transformed into heat, electricity and/or fuel.

### TECHNICAL BACKGROUND

Cellulose molecules mainly make up the cell walls of most plants. The invention relates to a new composition based on iron and a superabsorbent polymer intended to increase methane production yields by anaerobic fermentation, in particular from lignocellulose materials of plant origin.

The well-known methanization process takes place anaerobically. Organic matter decomposes by the presence of many species of bacteria. This reaction occurs in a sealed tank, called a digester, in which organic waste is stored to be subjected to the action of micro-organisms (bacteria) in the absence of oxygen. The main stages that occur during fermentation are:
hydrolysis and acidogenesis: acidogenic microorganisms transform complex organic chains into simpler compounds: peptides, amino acids, fatty acids, sugars;
acetogenesis: the products of acidogenesis are converted into acetic acid;
methanogenesis: methanogenic microorganisms are responsible for the production of gas (gasification): the acetic acid obtained during acetogenesis is transformed into methane and carbon dioxide.

Methane fermentation therefore allows energy recovery from organic matter. Anaerobic digestion produces on average 3 times less CO2 than classic aerobic fermentation, so it is a very efficient source of renewable energy. Biogas produced by methanization can replace natural gas, for example to produce heat, electricity and/or fuel for vehicles. The quantity of biogas generated is representative of the quality of fermentation. When this is well controlled, 1 kg of fermented sugar leads to a production of 600 liters of biogas composed essentially of methane CH4 (generally >60 v/v) and carbon dioxide CO2. Other elements may also be present in very small proportions. The calorific value (PCI) of biogas depends on the proportion of methane; for example, for biogas containing 65% methane, the PCI will be 6.46 kWh/m3.

What's more, once methanized, the residual material (digestate) is easily recyclable, particularly in the form of fertilizer because it is mainly made up of ammonia, a product of the transformation of the nitrogen which was contained there before fermentation.

According to some theories, the bacteria involved in anaerobic digestion could have been the first living organisms to appear on Earth, 3 billion years ago, when there was no oxygen in the atmosphere. Just like today, they degraded the organic molecules present (CO2 and hydrogen) into methane and oxygen. Biogas-producing bacteria could therefore be at the origin of the appearance of oxygen on Earth and, consequently, of life. The British H. Davy demonstrated the presence of methane in the gases produced during the decomposition of slurry as early as 1808. Nearly 100 years later, in 1897, the first digester was built in India with the aim of producing vehicle fuel.

The current sector is mainly based on the use of methanization processes which include the introduction of organic materials, typically liquid agricultural effluents (particularly slurry) to which other wastes (called co-substrates or inputs) are added and which can go up to 40% dry matter. The first provide the water and the microorganisms ensuring the methanization reactions, the second the material with the highest biogas yield. The methanization process consists of transporting the organic materials to be treated, most often by means of pumping systems, a hopper or an endless screw, inside a digester. The organic materials are then stirred continuously by one or more agitators in order to avoid the phenomena of settling, flotation or crusting of the biomass.

The influence of temperature is decisive for the proper functioning of fermentation. In fact, digesters are generally heated. The most frequently used fermentation, called mesophilic, takes place at around 35° C. There is also thermophilic fermentation (50-60° C.) which makes it possible to reduce the size of the methanizers as well as a better elimination of pathogenic germs. A two-step solution is also sometimes used: a first thermophilic reactor with a short residence time followed by a second mesophilic reactor.

The organic matter remains in the digester for a period of several weeks. The solid organic materials supplied are often crushed before being incorporated into the digestion tank in order to facilitate their transport and mixing. These processes require a significant expenditure of energy to be stirred continuously inside the digester.

The supply of organic matter is done regularly (one or several times a day) through feeding levels in order to preserve optimal physicochemical conditions for methanogenic activity (temperature, pH). This progressive addition of solid cosubstrate in the digester results in a phenomenon of accumulation of organic matter which is closely linked to its degradation rate. To control this phenomenon, it is often necessary to pre-treat the solid organic matter before its introduction, for example by grinding the solid part and removing (sorting) as much of the undesirable material as possible.

Furthermore, in order to allow mechanical stirring, in certain processes, the solid content of the reaction medium is fixed and must therefore not exceed 10 to 15%.

### Presentation of the Invention

Despite its many advantages, the methanization process still requires improvement in its efficiency and robustness. In particular, lignocellulosic residues (based on cellulosic and/or hemicellulosic fibers and lignin) are among the slowest to be degraded during anaerobic digestion. Thus, the anaerobic biodegradation of lignocellulosic residues, such as straws, generally requires residence times of 40 days or more in the digester, which has the consequence of significantly reducing its energy yield. This is the case, in particular for cereal straws, which greatly slows down their recovery through methanization.

To date, the main solutions proposed to resolve this problem are based on:
sorting organic matter before it is introduced into the digester,
physical and/or chemical processes leading to the destructuring of the lignocellulosic matrix,
and biological pretreatments with specific enzymes or microorganisms.

All are relatively expensive in terms of time, price and/or energy. There is therefore still an unmet need which would help in the degradation of this lignocellulosic biomass directly in the anaerobic digesters and without generating additional costs for the methanizer operator.

Recently, patent application FR19004513, filed by the applicant, demonstrated that the regular addition of at least one superabsorbent polymer, at a very low dose, during the addition(s) of organic matter into the methanizer makes it possible to increase effectively degrades lignocellulosic residues present in the digester, thus reducing their residence time.

The present invention relates to an improved composition based on—at least one superabsorbent polymer—and iron making it possible, by synergistic effect, to increase and/or further accelerate the anaerobic degradation of organic residues, in particular ligno-cellulosics, particularly with a view to their energy recovery by methanization.

The use of iron is well known in anaerobic digestion, mainly to eliminate hydrogen sulphide (H2S) present in the biogas before it is sent to the recovery module (cogeneration, injection) because this gas is partly responsible for the deterioration of the pipes and engines. The sulfur-reducing bacteria (SRB) present in the digester are responsible for the production of H2S from all forms of sulfur compounds. H2S is not very toxic for methanogenic bacteria, but it allows SBR to compete with them for the use of acetic acid. This results in a reduction in the quality of the biogas. It is therefore sometimes necessary to limit the production of H2S by a sulphide elimination reaction in the presence of iron. This reaction is written as follows:

2Fe³⁺+3H₂S→2FeS+S+6H⁺Fe²⁺+H₂S→FeS+2H⁺

In the context of the present invention, it has been found that the joint use of a superabsorbent polymer and iron, even at a very low iron concentration not allowing a reduction in hydrogen sulfide, leads to a synergistic action, which makes it possible to unexpectedly and very significantly increase the degradation of lignocellulosic residues present in an anaerobic digester, thus boosting methane production.

### INVENTION'S SUMMARY

The inventors have discovered, surprisingly, that the regular addition, preferably daily, of a composition comprising a superabsorbent polymer and iron, preferably in the form of ferrous or ferric salts, added hydrated, at a very low dose, separately or mixed when adding solid organic matter makes it possible to effectively and very simply increase the degradation of lignocellulosic residues present in the digester, thus reducing their residence time.

The invention thus allows operators not only to overcome a major technical problem but also to make substantial savings thanks to the gain generated in biogas production and to valorize by fermentation an abundant and available biomass which does not enter into competition with human or animal food.

### DETAILED DESCRIPTION OF THE INVENTION

A first aim of the invention is to propose a new composition, in this case a mixture in powder form comprising a superabsorbent polymer and iron, preferably in the form of ferrous or ferric salts, making it possible to accelerate the biodegradation process of my organic matter in anaerobic digesters, particularly with a view to optimizing methane production processes.

Another aim of the invention is also to propose a process for treating waste of organic origin, in particular agricultural, urban and agro-industrial, comprising lignocellulosic residues by using this composition to facilitate or accelerate their digestion in methanizers.

The present invention therefore relates to the joint use of a superabsorbent polymer and iron, preferably in the form of a mixture, to accelerate the fermentation of waste of organic origin, in particular agricultural, urban and agro-industrial, comprising a source of lignocellulosic residues, straw type, woody residues, etc. This composition is characterized in that:
the superabsorbent polymer is a water-retaining polymer, of natural or synthetic origin which has a water retention capacity greater than or equal to 10 times its weight in demineralized water, preferably greater than or equal to 20 times, advantageously greater than or equal to 30 times,
the iron is preferably present in the composition in the form of a water-soluble salt such as ferrous or ferric salts,
and the mass ratio between the superabsorbent polymer and the iron (SAP/Fe) is between 50 and 2000, preferably between 150 and 1000.

In the context of the invention, the type of water-retaining polymer, having a water retention capacity greater than or equal to 10 times its weight in demineralized water, preferably greater than or equal to 20 times, advantageously greater than or equal to 30 times, is generally known under the name of superabsorbent or by the abbreviation: SAP (“superabsorbent polymer”).

It generally comes in the form of powder, agglomerated or not.

Their structure based on a three-dimensional network comparable to a multitude of small cavities, each of which has the capacity to deform and absorb water, gives them the property of absorbing very large quantities of water and therefore swelling.

The superabsorbent polymers of natural origin, usable in the context of the present invention, are for example those described in patents U.S. Pat. No. 358,364, U.S. Pat. Nos. 1,693,890, 3,846,404, 3,935,099 or U.S. Pat. No. 3,661,815, etc. We will cite, without limitation: guar gum, alginates, carboxymethyl cellulose, dextran, xanthan gum, etc. The SAPs of synthetic origin that can be used in the context of the present invention are, for example, water-soluble polymers that are crosslinked, or can be crosslinked. There are many types. Such polymers are for example described in patent FR 2559158 in which crosslinked polymers of acrylic or methacrylic acid are described, crosslinked graft copolymers of the polysaccharide/acrylic or methacrylic acid type, crosslinked terpolymers of the acrylic or methacrylic acid type/acrylamide/sulfonated acrylamide and their alkaline earth or alkali metal salts. In a preferred embodiment, the monomers used for the preparation of the superabsorbent polymers are chosen from acrylamide and/or partially or totally salified acrylic acid and/or partially or totally salified ATBS (acrylamido tertio butyl sufonate) and/or or NVP (N vinylpyrrolidone) and/or acryloylmorpholine and/or partially or totally salified itaconic acid. In a preferred embodiment, the superabsorbent polymers are crosslinked homopolymers or copolymers based on partially or totally salified acrylic acid. Other hydrophilic monomers, such as for example cationic monomers, but also monomers with hydrophobic characteristics, could be used to produce superabsorbent polymers. Among the cationic monomers, mention will be made, by way of example, of diallyldialkyl ammonium salts and monomers of the dialkylaminoalkyl (meth)acrylate, dialkylaminoalkyl (meth)acrylamide type as well as their quaternary ammonium or acid salts. Particular mention will be made of quaternized or salified dimethylaminoethyl acrylate (ADAME) and/or dimethylaminoethyl methacrylate (MADAME), acrylamidopropyltrimethylammonium chloride (APTAC) and/or methacrylamidopropyltrimethylammonium chloride (MAPTAC). Synthetic superabsorbent polymers are generally crosslinked with 100 to 6000 ppm (parts per million) of at least one crosslinking agent chosen from the group comprising acrylic compounds such as for example methylene bis acrylamide, allylic compounds such as for example tertra allylammonium chloride, vinyls such as divinyl benzene, diepoxy, metal salts, etc. Some can also have double crosslinking, for example with an acrylic crosslinker. The superabsorbent polymers of the invention may also be post-treated by post-crosslinking the surface of the polymer particles in order to increase their absorption capacity under the effect of pressure as described for example in the applications patent DE 4020780 C1, DE 19909653 A1 and DE 199098838 A1.

For cost reasons, absorbent materials of synthetic origin of the crosslinked sodium or potassium acrylate (co) polymer type with or without post-crosslinking will be preferred.

SAP can be obtained by all polymerization techniques well known to those skilled in the art: gel polymerization, precipitation polymerization, emulsion polymerization (aqueous or inverse) followed or not by a distillation step, suspension polymerization, polymerization in solution, these polymerizations being followed or not by a step making it possible to isolate a dry form of the (co) polymer by all types of means well known to those skilled in the art. The absorbent materials mentioned above can also be combined with each other.

The iron, used according to the invention, is preferably added in the form of a ferrous or ferric salt soluble in water. Indeed, depending on their oxidation number, metals are more or less soluble in water. Thus, ferrous iron is much more soluble than ferric iron. In chemistry, the ferrous ion (Fe2+) is the divalent ion of iron (+II oxidation state), as opposed to the ferric ion (Fe3+), which indicates a trivalent iron compound (oxidation state+III). Among the water-soluble iron II and/or III salts known to those skilled in the art, mention may be made, without limitation, of chloride, sulfate, citrate, malate, glycerophosphate, lactate, aspartate, gluconate, fumarate of iron and their derivatives as well as iron complexes such as for example based on glycinate or bisglycinate. A mixture of several different iron salts can also be used.

In the context of the invention, the SAP and the iron are preferably presented in the form of a homogeneous mixture in order to be introduced simultaneously. This is a true “composition”. The composition of the invention is thus in solid, dry form, namely a powder, which results either from a mixture of powders or from the coating of the SAP using a solution of iron salts which can, if necessary, integrate a liquid binding agent well known to those skilled in the art (e.g.: oil, polyethylene glycol, etc.) in order to allow the iron powder to better adhere to the surface of the superabsorbent polymer. The mass ratio between the superabsorbent polymer and the iron (SAP/Fe) is between 50 and 2000, preferably between 150 and 1000.

From an operational point of view, the quantity of composition, based on a superabsorbent polymer and iron, brought into the digester each day will depend on the size of the latter as well as the quantity of organic ligno-cellulosic matter added. Ideally, it should be between 10 g and 500 g per m3 of addition of daily organic cosubstrate in the digester (i.e. between 0.01 g/L and 0.5 g/L, preferably between 0.05 g/L and 0.2 g/L). The use of a higher quantity of composition is possible without affecting the fermentation, however the economic interest will be impacted.

According to the invention, the composition based on a superabsorbent polymer and iron is added to the digester in prehydrated form, namely that it will have been brought into contact with water so that the superabsorbent polymer is hydrated at most close to its maximum water retention capacity (which varies depending on the superabsorbent used and the hardness of the water). We have, indeed, shown that the greater SAP swelling, the better the methane production.

The final composition before injection into the digester therefore resembles a “jelly” and is composed of SAP swollen with “ferrous water”. The injection can be done as a mixture or, preferably independently before or after the introduction of the cosubstrate. It is carried out by regular feeding stages, preferably several times a week and advantageously, one or more times a day in order to preserve the optimal physico-chemical conditions for methanogenic activity (temperature, pH).

The present invention also relates to the process for the methanization of organic materials comprising ligno-cellulosic residues, of the straw type, characterized in that it comprises a step of bringing said ligno-cellulosic residue into contact, in an anaerobic environment, with a composition comprising at least one prehydrated superabsorbent polymer and iron as described above, said treatment leading to an increase in the degradability of said lignocellulosic residues present in the digester, thus reducing their residence time in the latter.

The invention thus makes it possible to solve a major technical problem but also to improve the performance of digesters by increasing the production of biogas. It has been observed that, in association with the composition of the invention, the separate addition of an antifoaming agent, well known to those skilled in the art such as for example rapeseed oil, in the methanizer makes it possible to better stabilize the performance of the digester taking into account the overactivity of the latter generated by the composition of the invention.

Ligno-cellulosic residues can be chosen from cereal straw such as wheat, corn, rapeseed, etc. and/or all types of woody residues (wood, miscanthus, etc.).

The invention thus makes it possible to contribute to the valorization of large quantities of lignocellulosic biomass currently poorly exploited (combustion sector) and as well as advantageously supplement the source of material to be digested, the latter being an inexhaustible resource. It also makes it possible to avoid or limit the use of so-called “food” biomass, potentially edible by humans, which is currently used for its energy potential.

The mechanism of the synergistic effect of the composition of the invention “SAP+iron” is not known. It has been observed that it is not based on a decrease in H2S production, with methane production increasing sharply even when the quantity of H2S produced remains constant. Without wishing to put forward any theory, the composition according to the invention could, for example, serve as a selective bioreactor for microorganisms specifically involved in the different stages of methanization.

The present invention also relates to any variation or adaptation which will become clear to those skilled in the art, if necessary by having recourse to a few routine tests.

In addition to the preceding description, the invention will be better understood with the aid of the examples which follow and which are given by way of illustration and not limitation.

### Examples

The objective of the examples which were carried out in pilot testing is to study, in a liquid way, the action and effect of the composition of the invention, called “Powertek” in the context of the examples, on the process of bio methanization.

All comparative tests were carried out using reactors and under strictly identical conditions.

Isolated CSTR (continuously stirred tank reactor) reactors were used to carry out the simulation test. The reactor has a total volume of 85 liters with a wet volume of 72 liters. Heating of the reactors is carried out by heating mats. The temperature is monitored by a “Pt-100” type probe.

The reactor is filled with an inoculum with optimal characteristics and in relation to the typology of the process and the characterization of the feed mix. Before starting the test, the inoculum is incubated for a few days, without feeding to minimize background production.

The simulation is carried out with parameters classically used for mesophilic methanization (38° C.) in the wet process (departure: 5% dry matter, pH: 8.2). The input/cosubstrate ration (⅔ manure, rich in straw+⅓ slurry) added daily is 1.2 liters or 1/60 of the wet volume of the reactor.

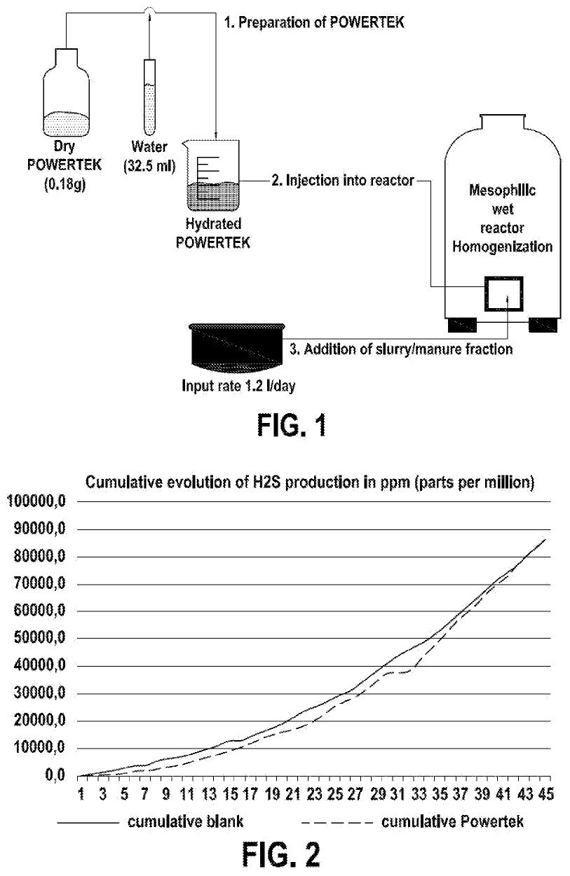

FIG. 1 represents a diagram of the protocol used to implement the tests.

Step 1: Hydration of the composition: 0.18 g (180 mg) of powder of the POWERTEK composition (SAP+FER) are taken, then hydrated with 32.5 ml of water and finally left to rest for 1 hour before the injection so that the activation (absorption of water by the superabsorbent polymer) is complete.

Step 2: Incorporation of the preparation: a full dose of the composition activated according to step 1 is injected every day into the methanizer (mesophilic wet process reactor) for the total duration of the pilot test.

Step 3: Incorporation of the input/cosubstrate ration (1.2 liters/day)

Table 1 below presents the variations in methane production obtained after 45 days,
counter-example 1 (CEx1): in the absence of the composition of the invention (=without steps 1 and 2 of the protocol)
and, according to the protocol described:
counter-example 2 (CEx2): as well as in the presence of the superabsorbent polymer alone (in accordance with patent application FR19004513 filed by the applicant).
   Example 1 (Ex1): in the presence of the composition of the invention

TABLE 1

| | | Mass ratio of the | |
|---|---|---|---|
| | | composition | Increase |
| | Preparation used: | between the | in methane |
| | commercial | superabsorbent | production after |
| | name/chemical nature | polymer and iron | 45 days compared |
| | (% anionicity) | (SAP/Fe) | to example 1 |
| CEx1 | na | na | na |
| CEx2 | Apromud G300 (100%) | na | +8.5% |
| | Sodium polyacrylate at a | (SAP used alone) | |
| | concentration of 0.15 g/L | | |
| | of input | | |
| Ex1 | POWERTEK composition: | (i.e. 180 mg of | +14.8% |
| | [Apromud G300: SAP | SAP/0.345 mg of | |
| | sodium polyacrylate | iron: mass of iron | |
| | (100%) + ferric chloride - | contained in 1 mg | |
| | FeCl₃] at a | of FeCl₃ | |
| | concentration of 0.15 g/L | | |
| | of input | | |

FIG. 2 presents the cumulative evolution of H2S production obtained in the absence and presence of the composition of the invention.

The examples described in Table 1 show that in the presence of the composition of the invention, compared to the counterexamples carried out without (Cex), the biomethanization of organic materials comprising ligno-cellulosic residues, of the straw type, carried out according to the process of the invention makes it possible to extremely significantly increase the production of methane (table 1). Table 2 demonstrates that the presence of iron at a concentration of 0.2875 mg per liter of input (cosubstrate) associated with SAP has no effect on H2S levels and that the overproduction of methane obtained thanks to the composition of the invention is not the consequence of a reduction in the production of H2S.

Unexpectedly, the use of iron associated with a superabsorbent polymer according to the invention increases the overproduction of methane by 74% compared to SAP used alone. This has the effect of reducing the residence time of substrates based on ligno-cellulosic residues, known to be among the slowest to be degraded during anaerobic digestion, and thus allows operators not only to overcome a technical problem major but also to make substantial savings thanks to the gain generated in biogas production.

It has been observed that the production of methane is so boosted by the composition of the invention that it may prove necessary to use, in association with the composition of the invention, an antifoam agent, well known by the skilled person, incorporated separately in the methanizer in order to better stabilize the performance of the digester taking into account the overactivity of the latter.

The invention thus makes it possible to contribute to the valorization of large quantities of lignocellulosic biomass (straw, wood fibers, etc.) currently poorly exploited, which is an inexhaustible resource.

It also makes it possible to avoid or limit the use of so-called “food” biomass, potentially edible by humans, which is currently used for its energy potential.

Other tests were carried out (same conditions) using different mass ratios between the superabsorbent and the iron (SAP/Fe=150 and 1000). They both led to a significant increase in methane production.

1. Composition for increasing and/or accelerating the anaerobic degradation of organic residues, of agricultural, urban and agro-industrial origin, with a view to their energy recovery by methanization, characterized in that it is in the form of a powder and comprises a mixture:
of at least one superabsorbent polymer, said superabsorbent polymer being chosen from the group of water-retaining polymers of natural or synthetic origin which has a water retention capacity greater than or equal to 10 times its weight in demineralized water, preferably greater than or equal to 20 times, advantageously greater than or equal to 30 times,
and iron.

2. Composition according to claim 1, characterized in that said iron present in the composition is in the form of a salt soluble in water and chosen from ferrous or ferric salts.

3. Composition according to claim 1, characterized in that the mass ratio between the superabsorbent polymer and the iron (SAP/Fe) is between 50 and 2000, preferably between 150 and 1000.

4. Composition according to claim 1, characterized in that said superabsorbent polymer comprises one or more monomers, chosen from the group of partially or totally salified acrylic acid monomers of the sodium or potassium acrylate (co) polymer type crosslinked with or without post crosslinking.

5. Process for the treatment by anaerobic degradation of organic waste of agricultural, urban and agro-industrial origin, in a digester with a view to their energy recovery by methanization, comprising ligno-cellulosic residues, characterized in that it comprises a step of implementation contact of said waste with a composition comprising at least one superabsorbent polymer and iron and characterized in that said composition is brought into contact with water before introduction into the digester so that the superabsorbent polymer is prehydrated, preferably as close as possible to its maximum water retention capacity.

6. Process according to claim 5, characterized in that the ligno-cellulosic residues are chosen from cereal straw and/or from woody residues.

7. Process according to claim 5, characterized in that the quantity of composition added to the digester is between 0.01 g/L and 0.5 g/L, preferably between 0.05 g/L and 0.2 g/L relative to the volumes of additions of organic cosubstrate, and is carried out as a mixture or, preferably independently before or after, the introduction of the cosubstrate, by regular supply stages, preferably several times per week and advantageously one or more times per day.

8. Process according to claim 5, characterized in that said iron present in the composition is soluble in water and chosen from ferrous or ferric salts.

9. Process according to claim 5, characterized in that the mass ratio between the superabsorbent polymer and the iron (SAP/Fe) present in the composition is between 50 and 2000, preferably between 150 and 1000.

10. (canceled)

11. The method of claim 5, wherein the superabsorbent polymer is chosen from the group of water-retaining polymers of natural or synthetic origin which has a water retention capacity greater than or equal to 10 times its weight in demineralized water, preferably greater than or equal to 20 times, advantageously greater than or equal to 30.
